(19) United States
(12) Patent Application Publication
(10) Pub. No.: US 2013/0005708 A1
(43) Pub. Date: Jan. 3, 2013

(54) HISTAMINE ANTAGONIST TREATMENT OF INFLAMMATORY SKIN DISORDERS

(76) Inventor: DINUSHA N. LALWANI, Corona, CA (US)

(21) Appl. No.: 13/537,181

(22) Filed: Jun. 29, 2012

Related U.S. Application Data
(60) Provisional application No. 61/502,563, filed on Jun. 29, 2011.

(51) Int. Cl. A61K 31/55; A61P 29/00; A61P 17/00

(52) U.S. Cl. 514/214.02

(57) ABSTRACT

Presented herein inter alia are compositions, methods, and kits for treating an inflammatory skin disorder using a histamine antagonist. In certain embodiments, the compositions, methods, and kits are for treating urticaria and its symptoms. In certain embodiments, the composition, methods, and kits include a topical pharmaceutical preparation with an effective amount of alcaftadine as an active agent.

### CROSS-REFERENCE TO RELATED APPLICATION

This application is based, and claims priority under 35 U.S.C. §120 to U.S. Provisional Patent Application No. 61/502,563 filed on Jun. 29, 2011, and which is incorporated herein by/reference.

### BACKGROUND OF THE INVENTION

Urticaria, commonly referred to as hives, is a common dermatologic disorder often manifesting as raised, pruritic, well-circumscribed areas of erythema and edema within the dermis and/or epidermis. In the United States, acute urticaria afflicts 15-20% of the general population at some time during their lives. Globally, the frequency of urticaria is similar to the United States.

Urticaria may be acute (lasting less than 6 weeks) or chronic (lasting more than 6 weeks). A large variety of urticaria variants exist, including acute immunoglobulin E (IGE) mediated urticaria, chemical induced urticaria (non-IGE mediated, urticarial vasculitis, autoimmune urticaria, cholinergic urticaria, cold urticaria, mastocystosis, muckle-wells syndrome and many others (Poonawalla, T., Kelly, B., Urticara: a review. Am J Clin Dermatol., 2009; 10(1): 9-21.

Urticaria may result from the release of histamine, bradykinin, leukotriene C4, prostaglandin D2 and other vasoactive substances from mast cells and basophils in the dermis. These substances cause extravasation of fluid into the dermis, leading to the urticarial lesions. The intense pruritus of urticaria may be the result of histamine released into the dermis. Histamine is the ligand for two membrane-bound receptors, the H1 and H4 receptors, which are present on many cell types. The activation of H1 histamine receptors on endothelial and smooth muscle cells leads to increased capillary permeability. The activation of H2 histamine receptors leads to arteriolar and venule vasodilation.

On physical examination, urticaria may be characterized by blanching, raised, palpable wheals, which can be linear, annular (circular) or arcuate (serpiginous). These lesions may occur on any skin area; they may be transient and/or migratory. These lesions are often separated by normal skin but may coalesce rapidly to form large areas of erythematous, raised lesions that blanch with pressure. Furthermore, hives associated with chronic urticaria generally last 4-36 hours (Kaplan, A. P., Urticaria and angioedema, In: Middleton E. Jr., Reed, C. E., Ellis, E. F., Adkinson, N. H., Jr., Yunginger, J. W., Busse, W. W., eds. Allergy: principles and practice, 5th ed. Vol 2., St. Louis Mosby-Year book, 1998L 1104-22). Current treatment strategies for management of urticaria may result in an undesirable sedative effect, thereby adversely affecting daily life.

The disability associated with urticaria has a serious impact on the lives of patients. Better methods for treatment for urticaria and other dermatologic disorders are needed. The compositions, methods, and kits presented herein provide solutions for this and other problems.

### BRIEF SUMMARY OF THE INVENTION

Presented herein inter alia are compositions, methods, and kits for treating an inflammatory skin disorder (e.g., urticaria) using a histamine antagonist (e.g., alcaftadine). In certain embodiments, the compositions, methods, and kits are for treating symptoms of urticaria and include a topical pharmaceutical preparation with an effective amount of alcaftadine as an active agent.

In one aspect, a method is provided for treating urticaria in a patient. The method includes administering a pharmaceutical preparation that includes an effective amount (e.g., a therapeutically effective amount) of alcaftadine.

In some embodiments, the urticaria is immunoglobulin E (IgE) mediated urticaria, chemical induced urticaria, urticarial vasculitis, autoimmune urticaria, cholinergic urticaria, cold urticaria, mastocystosis, or Muckle-Wells syndrome. In some embodiments, the urticaria is acute urticaria. In some aspects, the urticaria is chronic urticaria. In some embodiments, the urticaria is histamine-mediated urticaria.

In some embodiments, the administering is topically administering. In some embodiments, the administering is topically administering at or proximate to a physical presentation of the inflammatory skin disease (e.g., for urticaria, at or proximate to a urticarial wheal). In some embodiments, the pharmaceutical preparation is a topical pharmaceutical composition. In some embodiments, the topical pharmaceutical composition is a non-ophthalmic, epidermal pharmaceutical preparation.

In some embodiments, the topical pharmaceutical composition includes alcaftadine, a quarternary ammonium salt, an edetate salt, a phosphate salt, and water. In some embodiments, the quarternary ammonium salt is benzalkonium chloride. In some embodiments, the edetate salt is edetate disodium. In some embodiments, the phosphate salt is monobasic sodium phosphate. In some embodiments, the composition has a pH of about 7.

In certain embodiments, the effective amount of alcaftadine is about 0.25% w/v.

In another aspect, a non-ophthalmic epidermal pharmaceutical preparation is provided. The non-ophthalmic epidermal pharmaceutical preparation includes a histamine antagonist active agent (e.g., alcaftadine), a preservative, and a buffer. In some embodiments, the preservative is a quaternary ammonium salt. In some embodiments, the quaternary ammonium salt is benzalkonium chloride. In some embodiments, the buffer is a phosphate salt (e.g., monobasic sodium phosphate). In some embodiments, the preparation has a pH between about 6 and 8.5 (e.g., about 7). In some embodiments, the preparation further includes a chelating agent (e.g., an edetate salt, such as edetate disodium). In some embodiments, the pharmaceutical preparation is a topical gel, topical cream, or a transdermal patch.

In another aspect, presented herein is a kit for treating urticaria. The kit includes (i) a pharmaceutical preparation (e.g., an alcaftadine formulation) and (ii) instructions for treating the inflammatory dermatological disease (e.g., urticaria) by means of the pharmaceutical formulation. In some embodiments, the instructions for treating the disease include a description of the method set forth in any of the other aspects or embodiments. In some embodiments, the formulation is the formulation set forth in any of the other aspects or embodiments.

Some embodiments of the invention are included in the following paragraphs:

1. A method for treating urticaria in a patient in need thereof, said method comprising administering a pharmaceutical preparation, wherein said preparation comprises an effective amount of alcaftadine.

2. The method of paragraph 1, wherein said urticaria is immunoglobulin E (IgE) mediated urticaria, chemical induced urticaria, urticarial vasculitis, autoimmune urticaria, cholinergic urticaria, cold urticaria, mastocystosis, or muckle-wells syndrome.

3. The method of paragraph 1, wherein said urticaria is acute urticaria.

4. The method of paragraph 1, wherein said urticaria is chronic urticaria

5. The method of paragraph 1, wherein said urticaria is histamine-mediated urticaria.

6. The method of paragraph 1, wherein said administering is topically administering.

7. The method of paragraph 1, wherein said administering is topically administering at or proximate to a physical presentation of said urticaria.

8. The method of paragraph 7, wherein said physical presentation is an urticarial wheal.

9. The method of paragraph 1, wherein said pharmaceutical preparation is a topical pharmaceutical composition.

10. The method of paragraph 9, wherein said topical pharmaceutical composition is a non-ophthalmic epidermal pharmaceutical preparation.

11. The method of paragraph 9, wherein said topical pharmaceutical composition comprises alcaftadine, a quarternary ammonium salt, an edetate salt, a phosphate salt, and water.

12. The method of paragraph 11, wherein said quarternary ammonium salt is benzalkonium chloride.

13. The method of paragraph 11, wherein said edetate salt is edetate disodium.

14. The method of paragraph 11, wherein said phosphate salt is monobasic sodium phosphate.

15. The method of paragraph 11, wherein said composition has a pH of about 7.

16. The method of paragraph 1, wherein said effective amount is about 0.25% w/v.

17. A non-ophthalmic epidermal pharmaceutical preparation comprising alcaftadine, a preservative, and a buffer.

18. The non-ophthalmic epidermal pharmaceutical preparation of paragraph 17, wherein said preservative is a quaternary ammonium salt.

19. The non-ophthalmic epidermal pharmaceutical preparation of paragraph 18, wherein said quaternary ammonium salt is benzalkonium chloride.

20. The non-ophthalmic epidermal pharmaceutical preparation of paragraph 17, wherein said buffer is a phosphate salt.

21. The non-ophthalmic epidermal pharmaceutical preparation of paragraph 20, wherein said phosphate salt is monobasic sodium phosphate.

22. The non-ophthalmic epidermal pharmaceutical preparation of paragraph 17, wherein said preparation has a pH between about 6 and 8.5.

23. The non-ophthalmic epidermal pharmaceutical preparation of paragraph 22, wherein said pH is about 7.

24. The non-ophthalmic epidermal pharmaceutical preparation of paragraph 17, wherein said preparation further comprises a chelating agent.

25. The non-ophthalmic epidermal pharmaceutical preparation of paragraph 24, wherein said chelating agent is an edetate salt.

26. The non-ophthalmic epidermal pharmaceutical preparation of paragraph 25, wherein said edetate salt is edetate disodium.

27. The non-ophthalmic epidermal pharmaceutical preparation of paragraph 17, wherein said non-ophthalmic epidermal pharmaceutical preparation is a topical gel, topical cream, or a transdermal patch.

28. A kit for treating urticaria, said kit comprising (i) a pharmaceutical preparation and (ii) instructions for treating urticaria by means of said pharmaceutical formulation, wherein said pharmaceutical formulation comprises alcaftadine.

29. The kit of paragraph 28, wherein said instructions for treating urticaria comprise a description of the method of any one of paragraphs 1 to 16.

30. The kit of paragraph 28, wherein said pharmaceutical formulation is the formulation of any one of paragraphs 17 to 27.

### DETAILED DESCRIPTION OF THE INVENTION

### I. Definitions

The terms “a,” “an,” or “the” as used herein not only include aspects with one member, but also aspects with more than one member. For example, an embodiment including “a buffer and a chelating agent” should be understood to present aspects with at least a second buffer, at least a second chelating agent, or both.

The term “about” as used herein to modify a numerical value indicates a defined range around that value. If “X” were the value, “about X” would generally indicate a value from 0.90X to 1.10X. Any reference to “about X” minimally indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.”

In formulations including an “additional,” “further,” or “second” component, the second component as used herein is chemically different from the other components or first component. A “third” component is different from the other, first, and second components, and further enumerated or “additional” components are similarly different.

“Agent” as used herein indicates a compound or mixture of compounds that, when added to a pharmaceutical formulation, tend to produce a particular effect on the formulation's properties. For example, a formulation including a thickening agent is likely to be more viscous than an otherwise identical comparative formulation that lacks the thickening agent.

“Alcaftadine” as used herein includes 2-(1-methylpiperidin-4-ylidene)-4,7-diazatricyclo[8.4.0.0^{(3,7)}]tetradeca-1(14),3,5,10,12-pentaene-6-carbaldehyde as well as its pharmaceutically acceptable salts, polymorphs, and prodrugs (e.g., an imine formed with a carrier amine; a biodegradable polymer comprising alcaftadine, such as a polyimide). In certain aspects, “alcaftadine” includes isotopically labeled analogs of alcaftadine (e.g., ¹³C-labeled alcaftadine). In certain aspects, “alcaftadine” includes homologs or analogs of alcaftadine with similar biological activity. In some embodiments, alcaftadine is 2-(1-methylpiperidin-4-ylidene)-4,7-diazatricyclo[8.4.0.0^{(3,7)}]tetradeca-1(14),3,5,10,12-pentaene-6-carbaldehyde as well as its pharmaceutically acceptable salts.

In general, embodiments described herein that include chiral compounds may include embodiments with the racemic form or embodiments enriched in the R- or S-enantiomer thereof (up to and including pure or substantially pure R— or S— enantiomer).

The terms “dose” and “dosage” are used interchangeably herein. A dose refers to the amount of active ingredient given to an individual at each administration. As used herein, the dose will generally refer to the amount of histamine antagonist or anti-inflammatory agent. The dose will vary depending on a number of factors, including the range of normal doses for a given therapy, the frequency of administration; size and tolerance of the individual; severity of the condition; risk of side effects; and the route of administration. One of skill will recognize that the dose can be modified depending on the above factors or based on therapeutic progress.

The term “dosage form” as used herein refers to the particular format of the pharmaceutical, and it depends on the route of administration. For example, a dosage form can be in a cream, gel, ointment, or patch, e.g., for topical or transdermal delivery; in a tablet or liquid, e.g., for oral delivery; or in a saline solution, e.g., for injection.

As used herein, the term “effective amount” or “effective dose” means an amount sufficient to achieve the desired result on the process or condition, and it accordingly will depend on the ingredient and the desired result. Nonetheless, once the desired effect is known, determining the effective amount is within the skill of a person skilled in the art.

“Formulation,” “composition,” and “preparation” as used herein are equivalent terms referring to a composition of matter suitable for pharmaceutical use (i.e., producing a therapeutic effect as well as possessing acceptable pharmacokinetic and toxicological properties).

An “inflammatory skin disease” as used herein is a disorder producing inflammation (or an inflammatory response) that manifests in the skin, often for a prolonged duration.

The term “non-ophthalmic epidermal” indicates the keratinized epithelial tissue of the skin's (e.g. human skin's) various layers (e.g., the dermis, epidermis, and associated tissue). It specifically excludes methods, compositions, and kits designed for use with the connective or epithelial tissue of the eyes (e.g., the sclera; the non-keratinized epithelial tissue of the cornea).

The term “non-ophthalmic epidermal pharmaceutical preparation” as used herein indicates a pharmaceutical composition directed to non-ophthalmic epidermal use.

The term “or” as used herein should in general be construed non-exclusively. For example, an embodiment of “a formulation including A or B” would typically present an aspect with a formulation including both A and B. “Or” should, however, be construed to exclude those aspects presented that cannot be combined without contradiction (e.g., a formulation pH that is between 9 and 10 or between 7 and 8).

Where a formulation is not aqueous, the term “pH” as used herein refers to the apparent pH of the formulation as determined by methods standard in the art.

As used herein, the term “pharmaceutically” acceptable is used as equivalent to physiologically acceptable. In certain embodiments, a pharmaceutically acceptable composition or preparation will include agents for buffering and preservation in storage, and can include buffers and carriers for appropriate delivery, depending on the route of administration.

As used herein, the term “physical presentation of the inflammatory skin disease” indicates a physiological symptom or feature of the disease. In certain embodiments, the inflammatory skin disease is urticaria. A common physiological symptom of urticaria is one or more urticarial wheals.

The term “prevent” as used herein refers to a decrease in the occurrence of dermatological symptoms (e.g., urticardial wheals) in a patient. The prevention may be complete (i.e., no detectable symptoms) or partial, so that fewer symptoms are observed than would likely occur absent treatment.

As used herein, the terms “prevent” and “treat” are not intended to be absolute terms. Treatment can refer to any delay in onset, e.g., reduction in the frequency or severity of symptoms, amelioration of symptoms, improvement in patient comfort, reduction in skin inflammation, and the like. The effect of treatment can be compared to an individual or pool of individuals not receiving a given treatment, or to the same patient before, or after cessation of, treatment.

As used herein, the term “proximate to a physical presentation of the inflammatory skin disease” indicates a site at or close to “a physical presentation of the inflammatory skin disease” as defined above. The site may border or be immediately adjacent to the physical presentation. It may also be nearby (e.g., within about 0.5, 1, 2, or 3 cm, if direct application to the physical presentation is painful).

The terms “subject,” “patient,” “individual,” and the like as used herein are not intended to be limiting and can be generally interchanged. That is, an individual described as a “patient” does not necessarily have a given disease, but may be merely seeking medical advice.

The term “subject” as used herein includes all members of the animal kingdom prone to suffering from the indicated disorder. In some aspects, the subject is a mammal, and in some aspects, the subject is a human.

The term “therapeutically effective amount” as used herein refers to that amount of the therapeutic agent sufficient to ameliorate one or more aspects of the disorder. For example, for the given aspect (e.g., length of incidence), a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

“Topical application;” “topical administration,” and “topically administering” are used interchangeably herein and include the administration of a composition to the skin, nail, mucosa, or other localized region of the body. In certain embodiments, the administration is to the skin. Topical application or administering may result in the delivery of an active agent to the skin, a localized region of the body, a localized volume of the body, or the systemic circulation. Topical application or administering is also meant to include the use of mouth washes and gargles.

“Topical formulation” and “topical pharmaceutical composition” are used interchangeably herein and include a formulation that is suitable for topical application to the skin, a nail, mucosa, or other localized region of the body. A topical formulation may, for example, be used to confer a therapeutic benefit to its user. Specific topical formulations can be used for topical, local, regional, or transdermal application of substances.

“Transdermal” as used herein includes a process that occurs through the skin. In certain embodiments, “transdermal” may also include epicutaneous, percutaneous, or transcutaneous.

“Transdermal application” or “transdermally administering” are used interchangeably herein and include administration through the skin. Transdermal application or administration can be used for systemic delivery of an active agent; however, it is also useful for delivery of an active agent to tissues underlying the skin with minimal systemic absorption. In certain embodiments, transdermal application or administration may also include epicutaneous, percutaneous, or transcutaneous application.

“Treatment” as used herein includes any cure, amelioration, or prevention of a disease. Treatment may prevent the disease from occurring; inhibit the disease's spread; relieve the disease's symptoms (e.g., fully or partially alleviate pain, itching, or inflammation from urticaria), fully or partially remove the disease's underlying cause, shorten a disease's duration, or do a combination of these things.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

As used herein, “urticaria” indicates a skin disorder often producing the symptom of urticarial wheals. In certain embodiments, the urticaria is hives.

As used herein, a “urticarial wheal” is used according to its plain ordinary meaning and generally refers to a pale or reddish area of skin. Urticarial wheals often are swollen or raised above the surrounding skin (e.g., a welt), and they usually are itchy (sometimes, severely itchy). In certain embodiments, the wheal has a distinct boundary separating affected skin from unaffected skin. In certain embodiments, the boundary between affected and unaffected skin is not distinct (e.g., the reddish patch is larger than the swollen area and may include several separate welts or raised areas).

### II. Embodiments

A. Active Agent

In one embodiment, the active agent is a histamine antagonist (e.g. alcaftadine). The histamine antagonist may have multiple targets, e.g., alcaftadine, which targets H1, H2, and H4 receptors; doxepin, which is targets H1, H2, and 5HT receptors. In another embodiment, the active agent is a combination of at least two histamine antagonists, which may be directed to the same or different histamine receptors. Administering a single composition containing two different histamine antagonists may permit the effective concentration of each active agent to be lower than if a single active agent is administered to the patient while still achieving the desired therapeutic effects. In some embodiments, the histamine antagonist is alcaftadine.

TABLE 1
Histamine Antagonist Comparison Chart

| Histamine Antagonist | Trade Name | Dose | Dispense |
|---|---|---|---|
| Fexofenadine | Allegra | 180 mg/day | Oral |
| Loratadine | Claritin | 10 mg/day | Oral |
| Cetirizine | Zyrtec | 10 mg/day | Oral |
| Hydroxine | Atarax | 10 mg 4times | Oral |
| Diphenylhydramine | Benadrl | 25 bid | Oral/Topical |
| Cyprohepatadine | Periacitin | 4 mg 4 times | Oral |
| Alcaftadine* | LASTACAFT ™ | | Topical/ |
| | | | Transdermal |
| Doxepin | Zonalon | Cream; qday | Topical |
| Calamine/ | Ivarest | Cream; qday | Topical |
| Diphenylhydramine | | | |

The actual amount of the compound to be administered in any given case will be determined by a physician taking into account the relevant circumstances, such as the severity of the condition, the age and weight of the patient, the patient's general physical condition, the cause of the condition, and the route of administration. A person skilled in the art will appreciate that the dosage and application area will vary and can be tailored to the area being treated (e.g., knees, fingers, toes, back, and the like).

In some embodiments, a composition permits delivery of a histamine antagonist in a daily dosage of about 0.01 mg to about 120 mg, preferably about 0.1 mg to 60 mg, preferably about 0.5 mg to about 30 mg, and most preferably about 1 mg to about 10 mg. Yet still more preferably, the formulation permits delivery of a daily dosage of about 0.5 mg, 1 mg, 2 mg, 3 mg, 4 mg, 5 mg, 6 mg, 7 mg, 8 mg 9 mg or 10 mg.

In some embodiments, the composition is at a concentration of the histamine antagonist (e.g. alcaftadine) from about 0.001 to 5 mg/mL (w/v). In some embodiments, the composition is at a concentration of active agent from about 0.01 to 5 mg/mL (w/v). In some embodiments, the composition is at a concentration of active agent from about 0.1 to 5 mg/mL (w/v). In some embodiments, the composition is at a concentration of active agent from about 1 to 3 mg/mL (w/v). In some embodiments, the composition is at a concentration of active agent from about 2.5 mg/mL (w/v). In some embodiments, the composition is at a concentration of active agent from about 0.001 to 1.50 mg/mL (w/v). In some embodiments, the composition is at a concentration of from about 0.01 to 0.75 mg/mL (w/v). In some embodiments, the concentration is from about 0.05 to 0.50, 0.10 to 0.35. 0.20 to 0.75, or 0.05 to 0.30 mg/mL (w/v). In some embodiments, the concentration is about 0.05, 0.06, 0.07, 0.08, 0.09, 0.10, 0.11, 0.12, 0.13, 0.14, 0.15, 0.16, 0.17, 018, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40 mg/mL (w/v).

In certain embodiments, the concentration of the histamine antagonist is such that the dosage amount can be provided by application of the composition from one to four times a day, such as one to two times a day, to the skin proximate to a physical presentation of the inflammatory skin disease. Alternatively, the composition can be applied to a skin area of about 1 to 10 cm², about 1 to 50 cm², about 10 to 200 cm², about 50 to 500 cm², or about 100 to 1000 cm².

In certain embodiments, the formulation provides a total or a systemic dose of the histamine antagonist that is less than 90%, 80%, 70%, 60%, 50%, 40% or 30% of the systemic daily dose of the maximum approved oral dose. In certain embodiments, the formulation less than 25%, less than 10%, or less than 5%, yet provides local or regional delivery levels sufficient for therapeutic benefit. In certain embodiments, the concentration is such that this dosage amount can be provided by application of the composition from one to four times, a day, such as one to two times a day. Alternatively, the composition can be applied to a skin area of about 1 to 10 cm², about 1 to 50 cm², about 10 to 200 cm², about 50 to 500 cm², or about 100 to 1000 cm².U

B. Urticaria and Other Inflammatory Skin Diseases

In certain embodiments, the compositions, methods, and kits presented herein are useful for treating an inflammatory skin disease (e.g., urticaria). In some instances, the inflammation can last weeks, months or even years. An extended duration of the inflammatory response is frequently provoked by a persistent stimulus of the inflammatory response. Chronic inflammation can be the result of progression of acute inflammation. Chronic inflammation can also ensue after repeat episodes of acute inflammation or can develop de novo.

A number of inflammatory illnesses have been found to be associated with persistent pathogen infection, irritant non-living foreign matter that cannot be removed by enzymatic breakdown or phagocytosis, or a “normal” tissue component that is recognized as non-self (a feature frequently associated with autoimmune diseases). The histological appearance of chronic inflammation frequently involves a mixed inflammatory cell infiltrate that is often associated with the presence of macrophages, lymphocytes and plasma cells with neutrophil and eosinophil polymorphs as possible minor components (neutrophil and eosinophil polymorphs are associated in greater numbers with acute inflammation).

Examples of inflammatory skin diseases include psoriasis, rosacea, scleroderma, and inflammatory dermatoses, such as eczema, atopic dermatitis, contact dermatitis (e.g., irritation produced by exposure to a chemical agent, such as nickel, latex rubber, or the oils produced by poison ivy), dermatitis heretiformis (e.g., skin inflammation associated with celiac disease), seborrhoeic dermatitis, nummular dermatitis (e.g., coin-shaped areas of skin inflammation), stasis dermatitis (e.g., irritation associated with fluid buildup in the lower body), perioral dermatitis, urticaria, and pruritus. In certain embodiments, the skin irritation associated with other inflammatory diseases can be treated using the methods of the invention. These include skin inflammation associated with systemic lupus erythematosis, systemic sclerosis or schleroderma, dermatomyosis, vasculitis, sarcoidosis, Behcet's syndrome, and the like. Additional inflammatory diseases are described, e.g., in Harrison's Principles of Internal Medicine, 12th Edition, Wilson, et al., eds., McGraw-Hill, Inc).

In some embodiments, the urticaria is acute (lasting less than 6 weeks, generally with evanescent wheals (i.e., with individual wheals lasting less than a day)) or chronic (lasting more than 6 weeks). In certain embodiments, urticaria can be acute immunoglobulin E (IGE)-mediated urticaria, chemical induced urticaria, non-IGE-mediated urticaria, urticarial vasculitis, autoimmune urticaria, cholinergic urticaria, cold urticaria, dermatological effects of Muckle-Wells syndrome, mastocystosis, and the like. In certain embodiments, the urticaria may be other varieties of urticaria, such as skin inflammation associated with angioedema, acquired C1 esterase inhibitor deficiency (i.e., urticaria produced by an enzyme deficiency), adrenergic urticaria (e.g., produced by strong emotion), anaphylaxis, aquagenic urticaria, exercise urticaria, galvanic urticaria, heat urticaria, physical urticaria (e.g., dermatographism), pressure urticaria, dermatological effects of Schnitzler syndrome, solar urticaria, systematic carpillary leak syndrome, urticarial allergic eruption, urticaria-like follicular mucinosis, and the like. See, e.g., Poonawalla, T., Kelly, B., Urticara: a review. Am J Clin Dermatol., 2009; 10(1): 9-21.

C. Pharmaceutical Preparations

In view of their useful anti-urticaria properties, the histamine antagonists (e.g., alcaftadine) described above may be formulated into various pharmaceutical forms for administration purposes. These compositions may be prepared by any of the methods well known in the art of pharmacy and drug delivery. For example, to prepare the compositions for treating urticaria, an effective amount of the active ingredient (e.g., in base or acid addition salt form, or alternatively, as the free compound) may be combined in intimate admixture with a pharmaceutically acceptable vehicle or medium, which may take a wide variety of forms depending on the form of preparation desired for administration. In general, the pharmaceutical compositions are prepared by uniformly and intimately bringing the active ingredient into association with a liquid carrier or a finely divided solid carrier or both, and then, if necessary, shaping the product into the desired formulation.

In certain embodiments, the present invention provides a composition with a non-ionic surfactant, wherein the non-ionic surfactant is in an amount of about 1% to about 15% w/w, such as about 1%, 2%, 3%, 4%, 5%, 6%, 7%, 8%, 9%, 10%, 11%, 12%, 13%, 14%, 15% w/v, and fractions in-between. In a preferred aspect, the non-ionic surfactant is in an amount of less than about 15%, 14%, 13%, 12%, 11%, 10%, 9%, 8%, 7%, 6%, 5%, 4%, 3%, 2%, or 1%.

Such non-ionic surfactants include, for example, a sorbitan fatty acid ester, a sorbitol fatty acid ester, a polyoxyethylene sorbitan fatty acid ester, polysorbate, a polyoxyethylene fatty acid ester, a polyoxyethylene alkyl ether, a polyoxyethylene hydrogenated castor oil derivative (PEGCastor oil), ora polyoxyethylene polyoxypropylene alkyl ether. In some embodiments, the non-ionic surfactants include polyoxyethylene (20) sorbitan monolaurate (Tween 20™) and polyoxyethylene (20) sorbitan monooleate (Tween 80™).

Other non-ionic surfactants include, for example, sorbitan monolaurate, sorbitan monopalmitate, sorbitan monostearate, sorbitan sesquistearate, polyoxyethylene sorbitan monolaurate, polyoxyethylene monopalmitate, polyoxyethylene sorbitan monostearate, polyoxyethylene sorbitan tristearate, polyoxyethylene sorbitan monooleate, polyoxyethylene sorbitan trioleate, polyoxyethylene sorbitol monolaurate, polyoxyethylene sorbitol hexastearate, polyoxyethylene sorbitol tetraoleate, polyoxyethylene lauryl ester, polyoxyethylene stearyl ester, polyoxyethylene oleyl ester, polyoxyethylene lauryl ether, polyoxyethylene cetyl ether, polyoxyethylene stearyl ether, polyoxyethylene oleyl ether, polyoxyethylene hexadecyl ether, propylene glycol monostearate, polyoxypropylene, orpolyoxyethylene cetyl ether.

The present composition may optionally include one or more of the following: glycerine, at least one antioxidant, a chelating agent, a preservative, a thickening agent, one or more emulsifiers, pharmaceutically acceptable formulation aids, and penetration enhancers. In certain embodiments, the penetration enhancers include, but are not limited to, ethyl alcohol, isopropyl alcohol, or octolyphenylpolyethylene glycol. In certain embodiments, the penetration enhancers include oleic acid, polyethylene glycol 400, propylene glycol, N-decylmethylsulfoxide, fatty acid esters (e.g., isopropyl myristate, methyl laurate, glycerol monooleate, and propylene glycol monooleate); N-methylpyrrolidone, and the like.

In certain embodiments (e.g., for transdermal delivery), the permeability of the stratum corneum is enhanced by treatment with a chemical permeability enhancer, such as dimethyl sulfoxide, decylmethyl sulfoxide, diethylene glycol monomethyl ether, diethyleneglycol monoethyl ether, sodium laurate, sodium lauryl sulfate, cetyltrimethylammonium bromide, benzalkonium chloride, lecithin (see, for example, U.S. Pat. No. 4,783,450), 1-n-dodecylazacycloheptan-2-one (see, for example, U.S. Pat. Nos. 3,989,816; 4,316,893; 4,405,616; and 4,557,934), ethanol, propanol, octanol, benzyl alcohol, lauric acid, oleic acid, valeric acid, isopropyl myristate, isopropyl palmitate, methylpropionate, ethyl oleate, propylene glycol, ethylene glycol, glycerol, butanediol, polyethylene glycol, polyethylene glycol monolaurate, urea, hydroxide (see, for example, U.S. Pat. No. 6,558,695), dimethylacetamide, dimethylformamide, 2-pyrrolidone, 1-methyl-2-pyrrolidone, ethanolamine, diethanolamine, triethanolamine, salicylic acid, citric acid, succinic acid, and permeability enhancing peptides (see, for example, U.S. Pat. No. 5,534,496).

In a preferred aspect, at least one molecular penetration enhancer is present in the carrier. In certain aspects, the MPE™ is selected from the group of terpenes, fatty acid esters, and fatty acid alcohols. More preferably, the MPE™ is a terpene. Examples include d-limonene, limonene oxide, geraniol, α-pinene, α-pinene oxide, thymol, menthone, menthol, neomenthol, 3-carene, l-carvol, carvone, carveol, 1,8-cineole (eucalyptol), citral, dihydrocarveol, dihydrocarvone, 4-terpinenol, fenthone, menthone, pulegone, pulegol, isopulegol, piperitone, camphor, terpineol, α-terpineol, terpinen-4-ol, linalool, carvacrol, trans-anethole, ascaridole, safrole, racemic mixtures thereof (e.g., DL-limonene), and pharmaceutically acceptable isomers thereof. In certain preferred aspects, a second MPE™ can be present (e.g., a fatty acid ester and a terpene).

In one specific embodiment, the composition of the present invention comprises limonene or geraniol. In one aspect, the composition comprises 0.1% to 5% (w/w) of limonene or geraniol such as 0.1, 1, 2, 3, 4 or 5%, and more preferably 3% to 5% (w/w).

In certain aspects, the terpene MPE™ can be included within an essential oil. Essential oils that include a substantial proportion of at least one terpene MPE™ include oils of peppermint, eucalyptus, chenopodium, anise, and yling-yling. Preferably, the essential oil is eucalyptus oil.

Alternatively, a fatty acid ester or fatty alcohol ester is used as an MPE™. Examples of preferred fatty acid ester MPE™s are glyceryl monoesters. More preferably, the MPE™ is glyceryl monolaurate. In one aspect, the composition comprises 0.5% to 5% (w/w) of glyceryl monolaurate such as 0.5, 1, 2, 3, 4 or 5%, and preferably 1% (w/w).

In another aspect, a fatty acid ester or a fatty alcohol ester is used as a permeation enhancer in the composition. Examples of fatty acid ester and fatty alcohol esters include butyl acetate, caproyl glycolate, cetyl lactate, cocoyl glycolate, decyl N,N-dimethylamino acetate, decyl N,N-dimethylamino isopropionate, diethyleneglycol oleate, diethyl sebacate, diisopropyl sebacate, dodecyl N,N-dimethylamino acetate, dodecyl N,N-dimethylamino butyrate, dodecyl N,N-dimethylamino isopropionate, dodecyl 2-(N,N-dimethylamino)propionate, EQ-5-oleyl ester, ethyl acetate, ethyl acetoacetate, ethyl propionate, glyceryl dilaurate, glyceryl dioleate, glycerol monoethers, glycerol monooleate, glycerol monolinoleate, isopropyl isostearate, isopropyl laurate, isopropyl linoleate, isopropyl myristate, isopropyl palmitate, isostearoyl glycolate, lauroyl glycolate, methyl acetate, methyl caprate, methyl laurate, methyl oleate, methyl propionate, methyl valerate, 1-monocaproyl glycerol, medium-chain-length monoglycerides, benzyl or substituted benzyl nicotinate, octyl acetate, octyl N,N-dimethylamino acetate, oleyl oleate, n-pentyl N-acetylprolinate, propylene glycol monolaurate, sodium lauroyl glycolate, tetradecyl N,N-dimethylamino acetate, tromethamine lauroyl glycolate and the like. Still other examples include sunscreens such as Padimate-O, homosalate, cinnamate esters, octocrylene, and the like.

Other permeation enhancers include fatty acids, lactic acid, fatty alcohols (e.g., oleyl alcohol, stearyl alcohol, decanol), fatty alcohol ethers, hexahydro-1-dodecyl-2H-azepin-2-one (e.g., laurocapram, Azone™) and derivatives thereof, dimethylsulfoxide (DMSO) and related sulfoxides (e.g., n-decyl methylsulfoxide), salicylic acid and alkyl esters thereof (e.g., methyl salicylate), N,N-dimethylacetamide, dimethylformamide, N,N-dimethyltoluamide, 2-pyrrolidinone and N-alkyl derivatives thereof (e.g., N-methyl-2-pyrrolidone (NMP) and N-octyl-2-pyrrolidinone), and 2-nonyl-1,3-dioxolane. See Osborne, D. W.; Henke, J. J. “Skin Penetration Enhancers Cited in the Technical Literature,” Pharmaceut. Tech. 58-66 (November 1997).

In certain aspects, the composition of the present invention comprises a preservative, such as propyl paraben or methyl paraben. In some embodiments, a composition can contain 0.001-8%, 0.01-6%, or 0.05-5% w/v of t a preservative or a combination of preservatives. A variety of preservatives are suitable, including, but not limited to, benzoic acid, benzyl alcohol, benzylhemiformal, benzylparaben, 5-bromo-5-nitro-1,3-diox-ane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, diethylene glycol dimethyl ether (“DEDM”) hydantoin, DEDM hydantoin dilaurate, dehydroacetic acid, dibromopropamidine diisethionate, 1,3-bis(hydroxymethyl)-5,5-dimethylimidazolidine-2,4-dione (“DMDM”) hydantoin, and the like.

In certain embodiments, the formulations may be (i) sterile or essentially free from microorganisms such as bacteria and viruses that can cause infection and (ii) optionally preservative-free.

In one aspect, the composition comprises a mixture of a water phase and an oil phase (e.g., a cream, gel, or emulsion). In some embodiments, the composition includes at least about 3, 5, 7, 9.5, 10, 10.5, 11, 11.5, 12, 14, 15, 20, 25, 30, 31, 31.5, 32, 32.5, 33, 35, 36, 37, 38, 39, 40, 41, 42, 43, 44, 44.5, 45, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 58, 59, 60, 61, 62, 63, 64, 65, 66, 67, 68, 69, 70, 71, 72, 73, 74, or 75% (w/v) of the oil phase. In some embodiments, the composition comprises at least about 35, 36, 37, 38, 39, 40, 41, 42, 43, 44, 44.5, 45, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 58, 59, 60, 61, 62, 63, 64, 65, 66, 67, 68, 69, 70, 71, 72, 73, 74, or 75% (w/v) of the oil phase. In some embodiments, the composition comprises at least about 50, 50.5, 51, 51.5, 52, 52.5, 53, 53.5, 54, 54.5, 55, 55.5, 56, 56.5, 57, 58, 59, 60, 61, 62, 63, 64, 65, 66, 67, 68, 69, 70, 71, 72, 73, 74, or 75% (w/v) of the oil phase.

In some embodiments, the composition comprises at most about 3, 5, 7, 9.5, 10, 10.5, 11, 11.5, 12, 14, 15, 20, 25, 30, 31, 31.5, 32, 32.5, 33, 35, 36, 37, 38, 39, 40, 41, 42, 43, 44, 44.5, 45, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 58, 59, 60, 61, 62, 63, 64, 65, 66, 67, 68, 69, 70, 71, 72, 73, 74, or 75% (w/v) of the oil phase. In some embodiments, the composition comprises at most about 35, 36, 37, 38, 39, 40, 41, 42, 43, 44, 44.5, 45, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 58, 59, 60, 61, 62, 63, 64, 65, 66, 67, 68, 69, 70, 71, 72, 73, 74, or 75% (w/v) of the oil phase. In some embodiments, the composition comprises at most about 50, 50.5, 51, 51.5, 52, 52.5, 53, 53.5, 54, 54.5, 55, 55.5, 56, 56.5, 57, 58, 59, 60, 61, 62, 63, 64, 65, 66, 67, 68, 69, 70, 71, 72, 73, 74, or 75% (w/v) of the oil phase.

In yet another aspect, the composition includes at least one additional pharmaceutically acceptable surfactant, such as a nonionic surfactant. In some embodiments, the surfactant is a polysorbate surfactant, such as polysorbate 20.

Other nonionic surfactants include (but are not limited to) cetomacrogol 1000, cetostearyl alcohol, cetyl alcohol, cocoamide diethanolamine, cocoamide monoethanolamine, decyl glucoside, glyceryl laurate, lauryl glucoside, polyoxyethylene ethers of fatty acids such as cetyl alcohol or stearyl alcohol, narrow-range ethoxylates, octyl glucoside, oleyl alcohol, poloxamers, polyethylene glycol, sorbitan monolaurate, polyoxyethylene sorbitan monolaurate, sorbitan dioleate, sorbitan trilaurate, sorbitan monopalmitate, polyoxyethylene (20) sorbitan monopalmitate, sorbitan monostearate, sorbitan tristearate, polyoxyethylene (20) sorbitan monostearate, sorbitan monooleate, sorbitan trioleate, polyoxyethylene (20) sorbitan monooleate, stearyl alcohol, sucrose coconut fatty ester mixtures, and sucrose monolaurate.

In still yet another aspect, the composition includes at least one additional thickening agent, such as a cellulosic thickening agent. Suitable cellulosic thickening agents include, but are not limited to, hydroxypropyl cellulose (HPC) of various grades, hydroxypropyl methyl cellulose, hydroxyethyl cellulose, hydroxyethyl methyl cellulose, ethyl cellulose, methyl cellulose, carboxymethyl cellulose, dextran, guar gum, pectin, starch, cellulose, and the like.

In an alternative or preferred aspect, the composition comprises 1% to 5% such as 1, 2, 3, 4, or 5% of a cellulosic thickening agent. More preferably, the composition comprises from 1% to 2% of a cellulosic thickening agent. Still more preferably, the composition comprises 1% of a cellulosic thickening agent. Alternatively, the composition comprises 2% of a cellulosic thickening agent.

In certain embodiments, the formulation may include antioxidants such as butylated hydroxytoluene (“BHT”), butylated hydroxyanisole (“BHA”), ascorbyl linoleate, ascorbyl dipalmitate, ascorbyl tocopherol maleate, calcium ascorbate, carotenoids, kojic acid, tocopherol, tocopherol acetate, tocophereth 5, tocophereth 12, tocophereth 18, tocophereth 80, and the like.

In certain embodiments, the formulation may include chelating agents such as ethylenediamine tetraacetic acid (“EDTA”), diammonium EDTA, dipotassium EDTA, calcium disodium EDTA, hydroxyethylethylenediaminetriacetic acid (“HEDTA”), ethylenediaminetetraacetic acid, mono(triethanolamine) salt (“TEA-EDTA”), tetrasodium EDTA, tripotassium EDTA, trisodium phosphate, diammonium citrate, galactaric acid, galacturonic acid, gluconic acid, glucuronic acid, humic acid, cyclodextrin, potassium citrate, the potassium salt of ethylenediamine-tetra(methylene phosphonic acid) (“EDTMP”), sodium citrate, sodium EDTMP, and the like.

The formulation's viscosity is a factor that determines how well the formulation sticks to the skin or does not run off the skin when applied. The viscosity of the formulation can be optimized using one or more pharmaceutically acceptable thickening agents that do not significantly interact with the components of the formulation, do not significantly reduce flux of the formulation, and do not cause stinging or irritation. In certain embodiments, one or more of the following thickening agents is used: polyacrylic acid polymers, carbomers, cellulose derivatives, poloxamers, poloxamines, dextrans, pectins, natural gums. In one embodiment, cellulose, hydroxyethyl cellulose (“HEC”), hydroxypropyl methyl cellulose (“HMPC”), carboxymethyl cellulose, and the like.

In yet another alternative aspect, the composition is more viscous than water at standard temperature and pressure (STP). Alternatively, the composition has a kinematic viscosity of more than about 1 centistokes (cSt) or a dynamic viscosity of more than about 1 centipoise (cP). In certain aspects, the dynamic viscosity of the composition is at least about 2, 3, 4, 5, 7, 10, 12, 15, 20, 25, 30, 35, 40, 45, 50, 60, 70, 75, 80, 90, 100, 150, 200, 250, 500, 1000, 2000, 3000, 5000, 10,000 cP at STP. In yet other aspects, the composition is thixotropic (i.e., it decreases in viscosity upon being stirred or shaken). The composition's viscosity can be adjusted by the addition of a cellulosic thickening agent, such as hydroxypropyl cellulose, or other thickening agents.

In one aspect, a non-ophthalmic epidermal pharmaceutical preparation is provided. In certain embodiments, the formulation pH may be too acidic or basic to be applied to the eye without irritation or pain. In certain embodiments, the formulation may include components (e.g., surfactants; penetration enhancers) that produce irritation or pain when topically applied to the eye. In certain embodiments, the formulation may include a penetration enhancing device (e.g., a patch) that cannot be applied to the eye because of excessive irritation or obstruction of vision.

It is often advantageous to formulate the aforementioned pharmaceutical compositions in dosage unit form for ease of administration and uniformity of dosage. Dosage unit form as used in the specification and claims herein refers to physically discrete units suitable as single-application dosages, each unit containing a predetermined quantity of active ingredient calculated to produce the desired therapeutic effect in association with the required pharmaceutical vehicle. Examples of such dosage unit forms are tablets (including scored or coated tablets), capsules, pills, powder packets, wafers, injectable solutions or suspensions, teaspoonfuls, tablespoonfuls and the like, and segregated multiples thereof.

These pharmaceutical compositions are desirably in a unitary dosage form suitable for administration topically, transdermally, orally, rectally, or by parenteral injection. Additionally, the formulations may be designed to delay release of the active compound over a given period of time, or to carefully control the amount of drug released at a given time during the course of therapy.

In preparing the compositions in oral dosage form, any of the usual pharmaceutical media may be employed, such as water, glycols, oils, alcohols and the like in the case of oral liquid preparations, such as aqueous or oily suspensions, emulsions, syrups, elixirs and solutions; or solid carriers, such as starches, sugars, kaolin, lubricants, binders, disintegrating agents and the like in the case of powders, granules, pills, hard or soft capsules, tablets, caplets, troches, and lozenges. Because of their ease in administration, tablets and capsules represent the most advantageous oral dosage unit form, in which case solid pharmaceutical carriers are employed.

Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically acceptable, attractive, and palatable preparations. Tablets may contain the active ingredient in admixture with non-toxic, pharmaceutically acceptable excipients which are suitable for the manufacture of tablets. These excipients may be, e.g., inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, such as corn starch, or alginic acid; binding agents, such as PVP, cellulose, PEG, starch, gelatin or acacia; and lubricating agents, such as magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time-delay material such as glyceryl monostearate or glyceryl distearate may be employed. They may also be coated to form osmotic therapeutic tablets for controlled release.

Formulations for oral use may also be presented as hard gelatin capsules wherein the active ingredient is mixed with an inert solid diluent, such as calcium carbonate, calcium phosphate or kaolin; or as soft gelatin capsules in which the active ingredient is mixed with water or an oil medium, such as peanut oil, liquid paraffin, or olive oil. Additionally, emulsions can be prepared with a non-water miscible ingredient, such as oils, and stabilized with surfactants, such as mono-diglycerides, PEG esters, and the like.

Aqueous suspensions contain the active materials in admixture with excipients suitable for the manufacture of aqueous suspensions. Such excipients include, e.g., suspending agents such as sodium carboxymethylcellulose, methylcellulose, hydroxypropylmethylcellulose, sodium alginate, polyvinyl-pyrrolidone, gum tragacanth and gum acacia; dispersing or wetting agents, such as a naturally-occurring phosphatide (e.g., lecithin), condensation products of an alkylene oxide with fatty acids (e.g., polyoxyethylene stearate), condensation products of ethylene oxide with long chain aliphatic alcohols (e.g., heptadecaethyleneoxycetanol); condensation products of ethylene oxide with partial esters derived from fatty acids and a hexitol (e.g., polyoxyethylene sorbitol monooleate); or condensation products of ethylene oxide with partial esters derived from fatty acids and hexitol anhydrides (e.g., polyethylene sorbitan monooleate). The aqueous suspensions may also contain a preservative (e.g., ethyl or n-propyl p-hydroxybenzoate) or a coloring agent. Aqueous suspensions for oral use may include a flavoring agents or a sweetening agent, such as sucrose or saccharin.

Oily suspensions may be formulated by suspending the active ingredient in a vegetable oil, such as arachis oil, olive oil, sesame oil or coconut oil, or in a mineral oil such as liquid paraffin. The oily suspensions may contain a thickening agent, such as beeswax, hard paraffin or cetyl alcohol. For an oral preparation, sweetening and flavoring agents such as those set forth above may be added to provide a palatable preparation. These compositions may also be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent, and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil; a mineral oil, such as liquid paraffin; or a mixture. Suitable emulsifying agents may be naturally-occurring gums, such as gum acacia or gum tragacanth; naturally-occurring phosphatides, such as soybean, lecithin, esters or partial esters derived from fatty acids and hexitol anhydrides (e.g., sorbitan monooleate); and condensation products of partial esters with ethylene oxide, such as polyoxyethylene sorbitan monooleate. Emulsions for oral use may also contain sweetening and flavoring agents.

In the compositions suitable for transdermal administration, the carrier optionally includes a penetration enhancing agent or a suitable wetting agent, which could optionally be combined with suitable additives of any nature in minor proportions. The additives may facilitate the administration to the skin or may be helpful for preparing the desired compositions. Such agents and additives would be included in proportions that would not produce a significant deleterious effect on the skin. These compositions may be administered in various ways, e.g., as a transdermal patch (e.g., an iontophoretic patch), a spot-on application, a spray (e.g., a solution), a foam, a gel, a cream, jelly, solution, suspension, or an ointment. Because of their increased water solubility over the corresponding non-salt form, acid or base addition salts of the subject compounds are often more suitable for the preparation of aqueous compositions. In certain embodiments, acid or base addition salts of the active agent would be used.

A mode of delivery via oral administration may make patients susceptible to potential adverse events such as sedation. In addressing urticaria, a transdermal or topical application will likely provide the advantage of faster relief and increased efficacy with less chance of undesired systemic effects.

In general, it is contemplated that an effective amount would be from about 0.001 mg/kg to 20 mg/kg body weight, such as from about 0.01 mg/kg to 5 mg/kg body weight.

In yet another aspect, the composition remains stable for an acceptable time period between preparation and use when stored in a closed container at normal ambient temperature. Preferably, an “acceptable time period” is at least about 30 days, at least about six months, at least about one year, or at least about two years.

In an alternative aspect, the present invention provides a formulation that degrades by less than 1% over the course of 6 months at room temperature. In some embodiments, the rate of degradation is less than 0.9, 0.8, 0.7, 0.6, 0.5, 0.4, 0.3, 0.2, or less than 0.1%, and all fractions in between, over the course of six months at room temperature.

In a preferred aspect, a formulation provides the advantage of favorable stability at six months, as reflected in the lack of any substantial changes in viscosity, the absence of phase separation and crystallization at low temperatures, and a low level of impurities.

In certain aspects, the compositions of the invention optionally include a buffer, a pH-adjusting agent, or an anti-oxidant. The topical formulations of the present invention may, for example, comprise a pH-adjusting agent. In some embodiments, the pH adjusting agent is a base. Suitable pH-adjusting bases include amines, such as diethanolamine, triethanolamine, or aminopropanol; bicarbonates; carbonates; and hydroxides, such as ammonium hydroxide, alkali or alkaline earth metal hydroxide, or transition metal hydroxides. Alternatively, the pH adjusting agent can also be an acid, an acid salt, or mixtures thereof.

In some embodiments, a small amount of acid or base is included in the formulation. Non-limiting examples of amounts of acid or base that may be included in the formulation are about 0.000001%, 0.00001%, 0.0001%, 0.001%, 0.0012%, 0.01%, 0.012%, 0.1%, or 1.0%. This amount may be about 0.0001%. 0.0002%, 0.0003%, 0.0004%, 0.0005%, 0.0006%, 0.0007%, 0.0008%, 0.0009%, 0.0010%, 0.0011%, 0.0012%, 0.0015%, 0.0016%, 0.0017%, 0.0018%, 0.0019%, 0.002%, 0.003%, 0.004%, 0.005%, 0.006%, 0.007%, 0.008%, 0.009%, 0.01%, 0.012%, or 0.02%. This amount may be about 0.001%. 0.002%, 0.003%, 0.004%, 0.005%, 0.006%, 0.007%, 0.008%, 0.009%, 0.010%, 0.011%, 0.012%, 0.015%, 0.016%, 0.017%, 0.018%, 0.019%, 0.02%, 0.03%, 0.04%, 0.05%, 0.06%, 0.07%, 0.08%, 0.09%, 0.1%, or as needed to adjust the formulation to the desired pH.

In certain embodiments, the pH of the composition of the invention can be adjusted or stabilized with a buffer. Suitable buffers include citrate/citric acid buffers, acetate/acetic acid buffers, phosphate/phosphoric acid buffers, formate/formic acid buffers, propionate/propionic acid buffers, lactate/lactic acid buffers, carbonate/carbonic acid buffers, ammonium/ammonia buffers, and the like. In certain embodiments, the buffer is an acidic buffer system, such as benzocaine, citric acid, or a citric acid salt. In certain embodiments, the buffer system comprises panthenol, either alone or in combination with 3-aminopropanol.

In certain embodiments, the buffer is present at a concentration of about 0.000001 M, 0.00001 M, 0.0001 M, 0.001 M, 0.0012 M, 0.01 M, 0.012 M, 0.1 M, or 1.0 M. In certain embodiments, this amount is about 0.0010 M, 0.0015 M, 0.002 M, 0.003 M, 0.004 M, 0.005 M, 0.006 M, 0.007 M, 0.008 M, 0.009 M, 0.01 M. 0.012 M, or 0.02 M. In certain embodiments, this amount may be about 0.001 M. 0.002 M, 0.003 M, 0.004 M, 0.005 M, 0.006 M, 0.007 M, 0.008 M, 0.009 M, 0.010 M, 0.011 M, 0.012 M, 0.015 M, 0.016 M, 0.017 M, 0.018 M, 0.019 M, 0.02 M, 0.025 M, 0.03 M, 0.035 M, 0.04 M, 0.045 M, 0.05 M, 0.055 M, 0.06 M, 0.065 M, 0.07 M, 0.075 M, 0.08 M, 0.085 M, 0.09 M, 0.095 M, or 0.1 M. In certain embodiments, this amount may be about 0.10 M, 0.11 M, 0.12 M, 0.13 M, 0.14 M, 0.15 M, 0.16 M, 0.17 M, 0.18 M, 0.19 M, 0.20 M, 0.21 M, 0.22 M, 0.23 M, 0.24 M, 0.25 M, 0.26 M, 0.27 M, 0.28 M, 0.29 M, 0.30 M, 0.31 M, 0.32 M, 0.33 M, 0.34 M, 0.35 M, 0.36 M, 0.37 M, 0.38 M, 0.39 M, 0.40 M, 0.41 M, 0.42 M, 0.43 M, 0.44 M, 0.45 M, 0.46 M, 0.47 M, 0.48 M, 0.49 M, 0.50 M, 0.55 M, 0.60 M, 0.65 M, 0.7 M, 0.75 M, 0.8 M, 0.85 M, 0.9 M, 0.95 M, or 1.0 M. In certain embodiments, the formulation includes a buffer, and a second pH-adjusting agent (e.g., sodium hydroxide or hydrochloric acid) to adjust the pH of the composition to a desired pH. In certain embodiments, the second pH-adjusting agent comprises two agents (e.g., sodium hydroxide and hydrochloric acid) that are included as needed to adjust the pH of the composition to a desired pH.

In some embodiments, the pH-adjusting agent is sodium hydroxide, hydrochloric acid, or a combination of both, and is present in an amount sufficient to adjust the pH of the composition to between about pH 4.0 to about 8.5, such as between about pH 5.5 to about 7.0, such as 6.0 or 6.5. In some embodiments, the pH is adjusted to about 4.0, 4.2, 4.4, 4.6, 4.8, 5.0, 5.2, 5.4, 5.6, 5.8, 6.0, 6.2, 6.3, 6.4, 6.6, 6.8, 7.0, 7.2, 7.4, 7.6, 7.8, 8.0, 8.4, 8.5, or any fraction in-between.

In another aspect, the formulation is acidic. In certain aspects, the formulation has a pH of below about 7.5, 6.5, 5.5, 4.5, 3.5, or even 2.5. In certain other aspects, the pH of the formulation may range from about 1.5 to 7, about 2 to 7, about 3 to 7, about 4 to 7, or about 5 to 7. In still other aspects, the pH of the formulation may range from about 1.5 to 5.5, about 2.5 to 5.5, about 3.5 to 5.5, or about 4.5 to 5.5. The formulation may include a buffering or pH-adjusting agent to maintain its acidic pH. In certain aspects, the formulation has a pH value between about 4 and 7, such as 4, 5, 6 or 7 and fractional values between 4 and 7.

In yet another aspect, the formulation is basic. In certain aspects, the formulation has a pH of above about 7, 8, 9, 10, 11, or 12. In certain other aspects, the pH of the formulation may range from about 7 to 12.5, about 7 to 11.5, about 7 to 10.5, about 7 to 9.5, or about 7 to 8.5. In still other aspects, the pH of the formulation may range from about 9 to 12.5, about 9 to 11.5, about 9 to 10.5, or about 8.5 to 10. The formulation may include a buffering or pH-adjusting agent to maintain its basic pH. In certain aspects, the formulation has a pH value between about 7 and 10 and fractional values between 7 and 10.

In still yet another aspect, the formulation is neutral. In certain aspects, the formulation has a pH of about 7. In certain other aspects, the formulation has a pH from about 6 to about 8.5, from about 5.5 to 8, about 6 to 8, about 6.5 to 8.5, or from about 6.5 to 7.5. The formulation may include a buffering or pH-adjusting agent to maintain its neutral pH. In certain aspects, the formulation has a pH value between about 6 and 8.5 and fractional values between 6 and 8.5.

In one aspect, the pharmaceutical composition is formulated as a cream, an emulsion, a gel (e.g., a hydrogel, an organogel, or an inorganic or silica gel), a lotion, a lacquer, an ointment, a solution (e.g., a highly viscous solution), or a transdermal patch. The pharmaceutical composition may also be prepared so that it may be applied to the skin as a foam. In one embodiment, the composition is a solution. Alternatively, the composition is a transdermal patch.

In certain aspects, the formulation can be any of the inventive formulations disclosed in the claims or specification of this application.

D. Transdermal Delivery

In some embodiments, the histamine antagonist or pharmaceutical composition my be formulated for transdermal delivery (administration). Human skin comprises the dermis and the epidermis. The epidermis has several layers of tissue, namely, stratum corneum, stratum lucidum, stratum granulosum, stratum spinosum, and stratum basale (identified in order from the outer surface of the skin inward). The stratum corneum presents the most significant hurdle in transdermal delivery of medications. The stratum corneum is typically about 10-15 micrometres thick, and it consists of flattened, keratised cells (corneocytes) arranged in several layers. The intercellular space between the corneocytes is filled with lipidic structures, and may play an important role in the permeation of substances through skin (Bauerova et al., Chemical enhancers for transdermal drug transport, European Journal of Drug Metabolism and Pharmacokinetics, 2001, 26(1/2): 85-94). The rest of the epidermis below the stratum corneum is approximately 150 micrometres thick. The dermis is about 1-2 mm thick and is located below the epidermis. The dermis is innervated by various capillaries as well as neuronal processes.

Transdermal administration of pharmaceuticals provides an alternative route of administration of medications without undesirable consequences associated with injections and oral delivery. See, e.g., U.S. Pat. Publ. Nos. 2004/0009180 and 2001/0046962. For example, needles often cause localized pain, and potentially exposes patients receiving injections to blood borne diseases. Oral administration may suffer from poor bioavailability of medications due to the extremely acidic environment of the patient's stomach. Transdermal administration techniques overcome these shortcomings by providing non-invasive administration of pharmaceuticals. It is desirable with transdermal administration to reduce any damage to a patient's skin. Thus, transdermal administration of medication may reduce or eliminate pain associated with injections, reduce the likelihood of blood contamination, and improve the bioavailability of drugs once they are incorporated systemically.

In some embodiments, transdermal administration improve the permeability of the stratum corneum. The transdermal therapy may be directed at administering pharmaceutical agents that are incorporated into a patient's circulatory system, and thus are systemically administered through the skin. Chemical enhancing agents may be used to increase the permeability of molecules through the skin. Mechanical apparatuses may be used to bypass or ablate portions of the stratum corneum. In addition, ultrasound or iontophoresis may be used to facilitate the permeation of pharmaceuticals through the skin. Transdermal administration may be used to deliver a pharmaceutical agent such as small molecules (e.g., alcaftadine) through the skin so that an agent may pass to the capillary bed in the dermis where it may achieve a therapeutic effect (e.g., a local effect, or, by broader dispersal, a regional or systemic effect).

The compositions of the invention may be used in an application device that permits application of the composition to a target site on the skin without applying the composition to non-target site areas of the skin. For example, a device may be employed that allows the composition to be applied without first applying the composition to one's fingers. Suitable devices include spatulas, swabs, syringes without needles, and adhesive patches. Use of an adhesive patch for transdermal delivery is also useful in the methods and compositions provide herein, aspects of which are described in more detail elsewhere. See, e.g., Tonnesen, P. et al., A double blind trial of a 16-hour transdermal nicotine patch in smoking cessation, New Eng J Medicine, 325(5); 311-315: August 1991. Use of spatulas, swabs, or the like may require the device to be inserted into a container containing the composition. Using syringes or adhesive patches may be accomplished by filling the syringe or patch with the composition. The composition may then be topically spread by the spatulas or swabs, or may be expelled from the syringes onto the person's skin.

In certain embodiments, the formulation may be an adhesive patch that can be placed onto the surface of the skin of the patient, where the patch comprises a polymeric carrier which can release a therapeutically effective amount of an active agent onto the skin surface of the patient. Application of an adhesive, polymeric patch can be preceded by pretreatment of the skin with ethanol wipes or dermal abrasion, and the patch can be used concurrently or in conjunction with a suitable permeation enhancement methodology such as iontophoresis.

Many specific embodiments of the delivery devices of the invention may be constructed by adapting intradermal and transdermal drug delivery technology and engineering principles. The adaptation may involve one or a combination of control of the active agent's concentration, control of solution viscosity and other flow properties, formulation of the active agent with additives which enhance (or inhibit) transdermal flux, controlling permeability rates through membranes (either by selection of membrane material, pore structure, or some combination of both), using microneedles of appropriate number and interior diameter, using drug coated or matrix embedded microstructures, using mechanisms for flow control, and the like, so as to achieve the desired flux rates, blood concentrations, and bioavailability profiles. Furthermore, specific devices may be designed for different patient groups. Thus the flux rates and profiles may differ for devices intended for groups of patients having different blood volumes and/or different metabolic rates. For example, an optimal flux rate profile for geriatric patient might be different from middle-aged adults, men may be different from women, children different from adults, and the like.

A formulated histamine antagonist solution may be delivered from a depot solution within the device to a skin interface member by means of, e.g., simple passive absorption or adsorption, hydraulic pressure-exerting means such as a spring; electrophoretic drive apparatus; or phonophoretic (ultrasonic) apparatus for impelling the solution from the device at a predetermined rate. In one embodiment, the delivery means may include a volume of the solution in contact with a back surface of a skin interface member (distal to the surface which contacts the skin), and the active agent's flux may be established by absorptive flow of the solution into the interface member together with diffusion from the interface member through the skin of the patient. In another embodiment, the solution is transported by capillarity or impelled flow through one or more micro-needles extending from the interface member to the intra dermal compartment of the patient's skin.

The device also may include a solution flow controller for regulating the flux rate of the active agent into the blood of the patient. This may take the form of an inherent property of the device, for example, one in which an amount of the histamine antagonist exposed to blood at the outset is high, so that the influx rate is high. As the active agent dissolves and is transported away from the site of application, less active agent is available for absorption and the flux drops, until too little agent is released to produce a therapeutic effect. In other embodiments, an active solution flow controller establishes a first, higher flux rate to establish a preselected active agent concentration within the blood of a patient and a second lower flux rate to maintain the concentration. Such an active device also may include a timer for triggering the flux rate change, or a solution-flow switch for initiating or terminating the flow of active agent into the blood of the patient.

In another embodiment, the interface member of the device comprises a microstructure (e.g. a microneedle or micropellet), or an array of microstructures, which penetrates the stratum corneum of the patient. The microstructure(s) may be coated with an amount of desmopressin sufficient to establish upon engagement with the skin a first, higher flux rate to establish a preselected low desmopressin concentration within the blood of a patient. Optionally, the microstructure(s) are fed by solution from the depot of a concentration and at a flow rate suitable for establishing and maintaining a desmopressin concentration within the desired low dose range. Alternatively, a solution flow controller which establishes a second, lower flux rate at a predetermined time to maintain the concentration thereby to produce a predetermined interval of substantially constant predetermined desmopressin concentration in the blood of the patient.

Transdermal delivery methods and devices can benefit from techniques that reduce the efficacy of the stratum corneum as a barrier to drug entry. These include, for example, mechanical methods for removing portions of the stratum corneum before applying a transdermal desmopressin delivery device. “Tape-stripping,” in which tape is pulled from the skin of the patient, removes skin cells and can increase skin permeability, but is painful and relatively uncontrolled. A more desirable method for removing the stratum corneum has been described in U.S. Pat. No. 5,441,490 (the complete disclosure of which is hereby incorporated by reference), in which suction is used to form a blister. Removal of the blister permits subsequent transdermal delivery without the interference of the stratum corneum, as described in Svedman et al. (1991) The Lancet 337:1506-1509. The skin can also be “micropunctured” to introduce “micropassages” or “microfissures” across the stratum corneum, to enhance subsequent transdermal delivery. Such devices and methods are described, for example, in U.S. Pat. Nos. 5,611,806; 5,843,114; 5,879,326; in U.S. Pat. Publ. No. US 2009/0042970; and in Wermeling et al. (2008) “Microneedles permit transdermal delivery of a skin-impermeant medication to humans,” PNAS 105(6):2058-2063. Once the stratum corneum has been punctured, stripped, or removed by suction, a transdermal delivery device can be applied to deliver low dose histamine antagonist with favorable pharmacokinetics.

Energy can also be used to ablate or otherwise increase the permeability of the stratum corneum. For example, electrodes can be used to generate micro-channels in the stratum corneum. A suitable device is described in U.S. Pat. No. 6,148,232, and its use to pre-treat skin prior to transdermal peptide administration (as a dried or lyophilized component of a printed “patch”) is described in U.S. Pat. No. 7,383,084. While lasers are also useful in ablating the stratum corneum to improve permeability, the public's limited access to medical lasers generally renders them less convenient than other skin permeabilization techniques.

E. Methods of Delivery

Presented herein are inter alia methods of treating urticaria, such as histamine-mediated urticaria. In certain embodiments, the methods include a low-dose transdermal application (or administration) or topical application (or administration) of alcaftadine (e.g., 0.25% w/v) for prevention and symptomatic control. Without being bound by any particular mechanistic theory, alcaftadine is an H1, H2 and H4 histamine receptor antagonist; decreases chemotaxis, and has been shown to demonstration eosinophil-activation inhibition. More specifically, anti-histamine-based formulations useful in treating urticaria may include H1/H2/H4 antagonist suspensions, such as those marked under the trade name Lastacaft™.

In some aspects, the methods for treating urticaria involve administering an alcaftadine formulation, such as an alcaftadine solution formulated at a concentration of 2.5 mg/ml (e.g., Lastacaft™, which is indicated for the prevention of itching associated with allergic conjunctivitis).

In certain aspects, the methods involve a therapeutically effective amount of alcaftadine used transdermally or topically, which will allow adequate absorption into the epidermal/dermal junction (i.e., a site of mast cell degranulation) to prevent further histamine release from mast cells and relieve symptoms associated with urticaria. Thus, in some embodiments, a non-ophthalmic epidermal pharmaceutical preparation is administered.

In some embodiments, a transdermal or topical application provides fast relief and increased efficacy with less chance of undesired systemic effects. In some embodiments, alcaftadine is applied transdermally near the site of physical presentation of urticarial wheals.

In some embodiments, the methods provided herein are useful in treating warm-blooded animals suffering from a dermatological disease (e.g., urticaria, angioedema) by administering to the warm-blooded animals an effective amount of a histamine antagonist.

Methods of transdermal delivery may include steps of disrupting the stratum corneum to reduce the impermeability of the stratum corneum, and applying an active agent to the skin location in which the stratum corneum has been disrupted. Disrupting the stratum corneum refers to either completely removing the stratum corneum from a region of a patient's skin, or partially removing portions of the stratum corneum at a location on the patient's skin so that relatively small stratum corneum-free regions of skin are present. The skin may be disrupted using any suitable method without imparting significant pain to the patient.

In some embodiments of the methods, the stratum corneum is non-chemically disrupted. For example, the stratum corneum may be abrasively scrubbed to disrupt the laminar barrier of the stratum corneum. In certain embodiments, the stratum corneum may be disrupted by applying an adhesive, such as adhesive tape or wax, to the skin, and subsequently removing the adhesive from the skin. Because such methods of disrupting the stratum corneum may cause some pain, it may be desirable to provide a topical anesthetic to the skin, such as lidocaine cream, to temporarily reduce any pain that may be caused by the disruption.

Additional transdermal methods that non-chemically enhance the skin's permeability include low frequency ultrasound (20 kHz to 1 MHz). Ultrasound is defined as sound at a frequency of between about 20 kHz and 10 MHz, with intensities of between 0 and 3 W/cm². Low-frequency ultrasound, as used herein, refers to ultrasound at a frequency that is less than 1 MHz, such as in the range of 20 kHz to 40 kHz. The ultrasound is delivered in pulses, for example, 100 msec pulses at a frequency of 1 Hz. The intensity of the ultrasound may vary between 0 and 1 W/cm², and frequently varies between 12.5 mW/cm² and 225 mW/cm². Typical duration of exposure to ultrasound is between about 1 and about 10 minutes. The ultrasound is applied without causing an increase in skin temperature greater than about 1 degree Celsius. Low frequency ultrasound may be used alone or in combination with the composition to improve the permeability of the skin to the neurotoxin. Examples of ultrasound techniques for improving skin permeability may be found in U.S. Pat. Nos. 6,002,961 and 5,814,599. Surprisingly, it has been discovered that low frequency ultrasound, when applied in conjunction with a composition containing a botulinum toxin, permeabilizes the skin but does not substantially alter the three dimensional conformation of the neurotoxin, such as purified botulinum toxin or botulinum toxin complexes. Thus, the bioactivity of the neurotoxin is maintained and the disorder is substantially treated.

Additionally, the ultrasound may be delivered prior to application of the active agent to the skin. Low-frequency ultrasound temporarily disrupts the stratum corneum so that subsequent topical application of an active agent may have greater efficacy. The disruption caused by the ultrasound persists for several minutes, for example between about 10 and 30 minutes, to provide easier transdermal delivery of an active agent to the patient. After about 30 minutes, the stratum corneum begins to resume its natural structure, and the permeability of the stratum corneum temporally decreases. In certain embodiments, the method includes the step of applying low frequency ultrasound to one or more regions of the skin and subsequently topically applying the active agent to those regions of the skin that were exposed to the low frequency ultrasound, where the active agent is provided in a composition containing an enhancing agent, which facilitates prolonged penetration of the active agent to the patient.

Additional approaches include iontophoresis which can help deliver the active agent to a subdermal target site by passing electrical current across a patch containing a composition comprising the active agent. In one embodiment, an electrode may be applied on the external surface of the transdermal patch, and a ground electrode is provided elsewhere on a patient's skin. A small direct current is applied through the electrode positioned on the transdermal patch to urge the botulinum toxin in the composition through the patient's skin. The amount of current is typically less than 1 mA/cm2, such as between 0.3 mA/cm² and 0.7 mA/cm². Because the effectiveness of transdermal delivery of the active agent through the skin is at least partially dependent on the polarity of the agent, it may be desirable to change the pH of the composition an impart a charge on the active agent to facilitate the effectiveness of the electrical current in transporting the agent through the skin (e.g., for alcaftadine, make the formulation more acidic or use a acid salt). Additionally, the current is passed through the electrodes for a time that does not permanently damage (e.g., burn) the skin. For example, the current may be passed for a period of time between about 1 minute and 15 minutes. For longer applications, it is desirable to pulse the current to reduce potentially damaging effects caused by the electricity.

The active agent (histamine antagonist) may be topically administered by any suitable method as determined by the attending physician. The methods of administration permit the active agent to be administered locally to a selected target tissue. Methods of administration include coating the skin with the composition so that the composition covers at least a portion of the target site. Administration methods also include applying a transdermal patch to the target site of the skin and causing the active agent in the transdermal patch to diffuse into the skin. For extended applications, adhesive patches are utilized so that the composition can slowly diffuse into the skin without repeated applications of the patch. For example, a patch may include a microprocessor that provides periodic release of active agent from the patch. Microprocessor patches may be especially advantageous in patches that have the microneedles or low frequency ultrasound devices, as discussed above. The microprocessor can provide a timed release of the composition depending on the particular condition being treated. An example of a microprocessor controlled pharmaceutical treatment device may be found in U.S. Pat. No. 6,334,856.

F. Kits

The preparations may, if desired, be presented in a bottle, jar, or other container-closure system approved by the Food and Drug Administration or other regulatory body, which may provide one or more dosages containing the active ingredient. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation as taught herein, a container closure system including the formulation or a dosage unit form including the formulation, and a notice or instructions describing a method of use as taught herein.

In some embodiments, the kit includes a container which is compartmentalized for holding the various elements of a formulation (e.g., the dry ingredients and the liquid ingredients), instructions for making the formulation, and instructions for treating the skin inflammation disease (e.g., urticaria). In certain embodiments, the kit may include the pharmaceutical preparation in dehydrated or dry form, with instructions for its rehydration (or reconstitution) and administration.

The examples are intended to illustrate and not to limit the scope of the present invention in all its aspects. Various modifications or changes in light thereof will be suggested to persons skilled in the art and are to be included within the scope of the appended claims. All publications, patents, and patent applications cited herein are hereby incorporated by reference in their entirety for all purposes.

What is claimed is:

1. A method for treating urticaria in a patient in need thereof, said method comprising administering a pharmaceutical preparation, wherein said preparation comprises an effective amount of alcaftadine.

2. The method of claim 1, wherein said urticaria is immunoglobulin E (IgE) mediated urticaria, chemical induced urticaria, urticarial vasculitis, autoimmune urticaria, cholinergic urticaria, cold urticaria, mastocystosis, or muckle-wells syndrome.

3. The method of claim 1, wherein said urticaria is acute urticaria.

4. The method of claim 1, wherein said urticaria is chronic urticaria

5. The method of claim 1, wherein said urticaria is histamine-mediated urticaria.

6. The method of claim 1, wherein said administering is topically administering.

7. The method of claim 1, wherein said administering is topically administering at or proximate to a physical presentation of said urticaria.

8. The method of claim 1, wherein said pharmaceutical preparation is a topical pharmaceutical composition.

9. The method of claim 8, wherein said topical pharmaceutical composition is a non-ophthalmic epidermal pharmaceutical preparation.

10. The method of claim 8, wherein said topical pharmaceutical composition comprises alcaftadine, a quarternary ammonium salt, an edetate salt, a phosphate salt, and water.

11. The method of claim 10, wherein said quarternary ammonium salt is benzalkonium chloride.

12. The method of claim 10, wherein said composition has a pH of about 7.

13. The method of claim 1, wherein said effective amount is about 0.25% w/v.

14. A non-ophthalmic epidermal pharmaceutical preparation comprising alcaftadine, a preservative, and a buffer.

15. The non-ophthalmic epidermal pharmaceutical preparation of claim 14, wherein said preservative is a quaternary ammonium salt.

16. The non-ophthalmic epidermal pharmaceutical preparation of claim 14, wherein said quaternary ammonium salt is benzalkonium chloride.

17. The non-ophthalmic epidermal pharmaceutical preparation of claim 14, wherein said buffer is a phosphate salt.

18. The non-ophthalmic epidermal pharmaceutical preparation of claim 17, wherein said phosphate salt is monobasic sodium phosphate.

19. The non-ophthalmic epidermal pharmaceutical preparation of claim 14, wherein said preparation has a pH between about 6 and 8.5.

20. The non-ophthalmic epidermal pharmaceutical preparation of claim 14, wherein said pH is about 7.
